(19) United States
(12) Patent Application Publication
(10) Pub. No.: US 2023/0082614 A1
(43) Pub. Date: Mar. 16, 2023

(54) USE OF CD44 ANTIBODY IN PREPARATION OF MEDICINE FOR TREATING PARKINSON'S DISEASE

(71) Applicant: NANTONG UNIVERSITY, Nantong, Jiangsu (CN)

(72) Inventors: Cheng SUN, Nantong, Jiangsu (CN); Yuejun WANG, Nantong, Jiangsu (CN)

(21) Appl. No.: 17/759,366

(22) PCT Filed: Sep. 5, 2021
(86) PCT No.: PCT/CN2021/116578

§ 371 Date: Jul. 22, 2022

(30) Foreign Application Priority Data
Dec. 16, 2020 (CN) 202011487656.2

(51) Int. Cl. C07K 16/28; A61P 25/16

(52) U.S. Cl. CPC C07K 16/2884; A61P 25/16; A61K 2039/505

(57) ABSTRACT

A CD44 antibody is used in preparation of a medicine for treating Parkinson's disease (PD). By means of a stereotactic injection method, the CD44 antibody (anti-CD44) is injected into the cortex part of PD model mice, and it is found that the antibody can significantly alleviate PD-related symptoms, such as improve the motor functions of the mice, restore the number of dopaminergic neurons in the substantia nigra part and inhibit neuroinflammation.

### TECHNICAL FIELD

The present invention relates to use of a CD44 antibody.

### BACKGROUND

Parkinson's disease (PD), also known as paralysis agitans, is the second most common neurodegenerative disease after Alzheimer's disease. According to statistics, 1 in every 800 people in the world suffers from PD. By 2030, due to the acceleration of the aging process, the prevalence of PD will double, and the number of patients is expected to exceed 9 million. Direct medical expenses of patients are expected to exceed USD10,000 per year, placing a heavy burden on families and society. The main clinical manifestations of PD are bradykinesia, static tremor, and rigidity of movement. The main pathological feature of PD is the reduction of dopaminergic neurons in the substantia nigra part. Dopamine-producing cells in the brain gradually lose their ability to affect the nervous system, limiting a patient's ability to control their muscles.

Clinical treatment for PD is mainly based on dopamine replacement therapy. Although the symptoms of PD patients can be improved to a certain extent, long-term use of this therapy can cause a variety of adverse reactions, such as anxiety, insomnia, hallucinations and other mental symptoms. Therefore, it is of great scientific significance and application value to further study the mechanism of the occurrence and development of PD and to find potential targets for the treatment of PD. At present, the pathogenesis of PD remains unclear and may be related to abnormal protein accumulation, inflammatory response, mitochondrial dysfunction, and oxidative stress. In recent years, neuroinflammation has received extensive attention as one of the important entry points of the pathogenesis of PD.

CD44 is a cell adhesion molecule, a main receptor of hyaluronic acid, and a main component of extracellular matrix, and is expressed on the surfaces of endothelial cells, hematopoietic stem cells, mesenchymal cells and tumor cells. CD44 is mainly involved in heterogeneous adhesion, that is, the adhesion of tumor cells to host cells and host matrixes. Heterogeneous adhesion plays a role in promoting tumor cell invasion and metastasis. In addition, CD44 acts as an inflammatory modulator of TLR4 co-receptor transduction to modulate activation of a Toll-like receptor (TLR).

### SUMMARY

### Technical Problem

An objective of the present invention is to provide use of a CD44 antibody in preparation of a medicine for treating PD with good effect.

### Technical Solution

Use of a CD44 antibody in preparation of a medicine for treating PD.

It is application in preparation of a medicine for treating PD by relieving motor dysfunction, improving MPTP-induced olfactory dysfunction, and increasing the expression of TH.

### Beneficial Effects

The present invention has good effect and a simple research method.

### BRIEF DESCRIPTION OF THE DRAWINGS

FIG. 1 is a schematic diagram showing that injection of a CD44 antibody can improve motor dysfunction in a PD model.

A: test procedure; B: rotarod test; C: tail suspension test; D: pole test; and E: olfactory test. MPTP: N-methyl-4-phenyl-1,2,3,6-tetrahydropyridine. *p<0.05, **p<0.01, ***p<0.001, ns: no statistical difference, one-way ANOVA analysis.

FIG. 2 is a schematic diagram showing that the CD44 antibody alleviates the loss of dopaminergic neurons in the substantia nigra part of PD model mice.

A: Morphological analysis of dopaminergic neurons in the substantia nigra part (by immunohistochemical analysis with a TH antibody). B: Statistical quantification. C: Analysis of TH protein expression in the substantia nigra part. The protein expression is analyzed by a western blot method. TH: tyrosine hydroxylase; and MPTP: N-methyl-4-phenyl-1,2,3,6-tetrahydropyridine. ***p<0.001, ns: no statistical difference, one-way ANOVA analysis.

FIG. 3 is a schematic diagram showing that the CD44 antibody alleviates neuroinflammation in the substantia nigra part of PD model mice.

A: Morphological analysis of microglia in the substantia nigra part (by immunofluorescence analysis with an IBA-1 antibody). B: Statistical quantification. C: Morphological analysis of astrocytes in the substantia nigra part (by immunofluorescence analysis with a GFAP antibody). D: Statistical quantification. IBA-1: ionized calcium binding adapter molecule 1; GFAP: glial fibrillary acidic protein; and MPTP: N-methyl-4-phenyl-1,2,3,6-tetrahydropyridine. ***p<0.001, ns: no statistical difference, one-way ANOVA analysis.

### DETAILED DESCRIPTION

Use of a CD44 antibody in preparation of a medicine for treating PD.

It is application in preparation of a medicine for treating PD by relieving motor dysfunction, improving MPTP-induced olfactory dysfunction, and increasing the expression of TH.

A method for studying the mechanism of action of a CD44 antibody in treating PD, including the following steps:

1. Antibody Injection

3-month-old B6 wild-type mice are anesthetized with phenobarbital (75 mg/kg). Unilateral intracerebral injection is performed by a stereotactic method at anteroposterior (AP) 0 mm and medial lateral (ML) −2 mm relative to the anterior fontanel and dorsal ventral (DV) −1.5 mm from the dural surface. Using a 10 μL microsyringe, 1 μL of Anti-CD44 (1 mg/ml) is injected into the mouse cerebral cortex at a rate of 200 nl/min. The control mice are injected with the same dose of immunoglobulin G (IgG) in the same way. After the injection, the needle is kept at the original site for 3 minutes to ensure absorption, and then the needle is slowly removed from the mouse brain and the scalp is sutured.

2. Preparation of a PD Animal Model

10 days after stereotactic injection, mice were induced to form PD model mice by intraperitoneal injection of a neurotoxin MPTP (N-methyl-4-phenyl-1,2,3,6-tetrahydropyridine). Within three days before administration, the mice were subjected to a series of behavioral training such as rotarod-climbing and pole-climbing. Mice injected with IgG and Anti-CD44 are divided into two groups. One group is the normal saline group and the other is the MPTP group. Then, the mice are modeled with MPTP (20 mg/kg/d) for one week, and then behavioral evaluation is performed. Compared with the control mice (injected with the same volume of normal saline), the mice with significant impairment of motor function are considered as successfully induced PD model mice and are used for subsequent tests.

3. Rotarod Test

A rotarod test is performed using an accelerated rotarod to detect motor coordination. The mice are placed on the rotarod in an accelerated mode (4-40 rpm) for 5 minutes, and the time of balance and continuous movement on the rotarod before falling is recorded. The test is repeated three times and the average is taken.

4. Tail Suspension Test

The rear ⅓ part of a mouse tail is fixed to a bar, making it 15 cm from the ground, and the time that the mouse is still within 10 min is counted.

5. Pole Test

An animal is placed on the apex of a vertically fixed pole (15 mm in diameter, 50 cm in length) with a rough surface, and the time it takes for the mouse to reach the bottom of the pole with the forelimbs from the apex is counted. The interval between two tests is 5 minutes, and the average value of 3 times of test is obtained.

6. Olfactory Test

A mouse is fasted for 20 hours in advance, and a clean cage is prepared and cheese is buried in five positions in order, namely the middle, upper left, upper right, lower left, and lower right parts, of clean bedding. The animal is put in the cage, and the time for the mouse to find the cheese is counted. If the mouse does not find the cheese within 300 s, the time is recorded as 300 s. Statistical analysis is performed after eliminating the minimum and maximum values.

7. Morphological Analysis of Dopaminergic Neurons in the Substantia Nigra Part

Tyrosine hydroxylase positive cells in the substantia nigra part of test mice are stained by a histochemical method, and the main operation steps are as follows: 1. the collected mouse brain tissue is placed in 4% paraformaldehyde and placed in a 4° C. refrigerator for 24 hours; 2. 1× PB is prepared with 20% and 30% sucrose for dehydration for 24 hours in turn, and if the tissue does not sink to the bottom, the time is extended appropriately; 3. the dehydrated brain tissue is sectioned, adjusted to a thickness of 12 μm, placed in an oven at 37° C. overnight, and stored at −20° C.; 4. the tissue sections are baked at 60° C. for 2 hours before staining; 5. the tissue sections are washed 3 times with PBS for 5 minutes each; 6. the tissue sections are blocked with a blocking buffer at 37° C. for 30 minutes; the tissue sections are washed 2 times with PBS, and blocked with an endogenous catalase blocker for 5 minutes; 7. the tissue sections are washed 2 times with PBS, blocked with a non-specific staining blocker for 30 min and incubated at 37° C. in an oven; 8. the tissue sections are incubated with a TH antibody (1:200) overnight at 4° C.; 9. the tissue sections are washed 2 times with PBS, and reacted with biotin-labeled goat-anti-mouse/rabbit IgG at 37° C. for 1 hour; 10. the tissue sections are washed 2 times with PBS, and reacted with streptavidin-peroxidase at 37° C. for 1 hour; 11. the tissue sections are washed 2 times with PBS, and subjected to DAB color development; 12. alcohol dehydration is conducted sequentially with 50% alcohol, 70% alcohol, 80% alcohol, 95% alcohol twice, and 100% alcohol twice, for 5 minutes each; and 13. the tissue sections are treated with anhydrous ethanol and xylene, transparentized with xylene thrice for 5 minutes each, and mounted with neutral resin. Then, the tissue sections are observed under a microscope and pictures are taken. The number of TH-positive cells is statistically analyzed with Image J software.

8. Preparation of a Protein Sample of the Substantia Nigra Part and Test of Expression Level

Formula of tissue lysis buffer: 25 mM of Tris-HCl, pH 7.4; 10 mM of NaF; 10 mM of Na₄P₂O₇; 2 mM of Na₃VO₄; 1 mM of EGTA; 1 mM of EDTA; 1% of NP-40; 10 μg/ml Leupeptin; 10 μg/ml Aprotinin; 2 mM of PMSF; and 20 nM of Okadaic acid. Homogenization is conducted with a desktop homogenizer (Polytron, PT2100). A sample is rotated and lysed at 4° C. for 1 hour, and centrifuged (at 13000 rpm, 4° C.) for 20 minutes. After centrifugation, the supernatant lipids are carefully removed, and the remaining supernatant is transferred to another centrifuge tube and centrifuged again. This process is repeated 2 times to completely remove lipids from the protein sample. The protein content of the sample is determined with a protein assay kit. According to the results, the protein concentrations of all samples are adjusted to the same level, a loading buffer is added, and the mixture is mixed uniformly, boiled at 100° C. for 5 minutes, and cooled to room temperature for western blot analysis.

The samples obtained in the above step are separated by polyacrylamide gel electrophoresis (SDS-PAGE), and the protein on the gel is transferred to PVDF membranes. The PVDF membranes after transfer are blocked with a Tris-buffered saline solution/Tween (TBST) buffer containing 5% bovine serum albumin for 1 hour at room temperature. The blocked PVDF is incubated with a primary antibody overnight (4° C.). After the reaction with the primary antibody, the PVDF membranes are washed three times with TBST, and then reacted with a secondary antibody for 1 hour at room temperature. After the reaction with the secondary antibody is over, the PVDF membranes are washed three times with TB ST. Finally, the PVDF membranes are reacted with a chemiluminescence assay system (Roche) and exposed to X-film (Kodak). Each quantification of the expression level of protein is analyzed with the software Quantity-One (Bio-Rad).

9. Morphological Analysis of Microglia and Astrocytes in the Substantia Nigra Part

Microglia and astrocytes in the substantia nigra part of test mice are stained by immunofluorescence, and the main operation steps are as follows: 1. the collected mouse brain tissue is placed in 4% paraformaldehyde and placed in a 4° C. refrigerator for 24 hours; 2. 1× PB is prepared with 20% and 30% sucrose for dehydration for 24 hours in turn; 3. the dehydrated brain tissue is frozen and sectioned, adjusted to a thickness of 30 μm, placed in an oven at 37° C. overnight, and stored at −20° C.; 4. the tissue sections are baked at 60° C. for 2 hours before staining; 5. the tissue sections are washed 3 times with PBS for 5 minutes each; 6. the tissue sections are blocked with a blocking buffer at 37° C. for 30 minutes; the tissue sections are washed 2 times with PBS, and incubated with an IBA-1/GFAP antibody (1:300) at 4° C. overnight; 7. the tissue sections are washed 3 times with PBS and incubated with a homologous secondary antibody for 3 hours at room temperature; and 7. after being washed 3 times with PBS, the tissue sections are mounted with a fluorescent mounting medium. Then, the tissue sections are observed under a microscope and pictures are taken. Fluorescence intensity is statistically analyzed with Image J software.

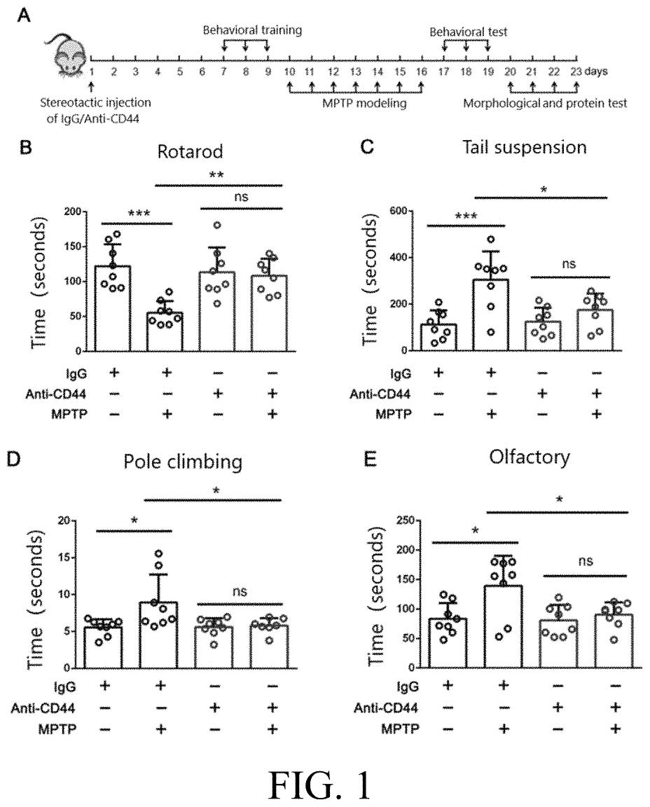

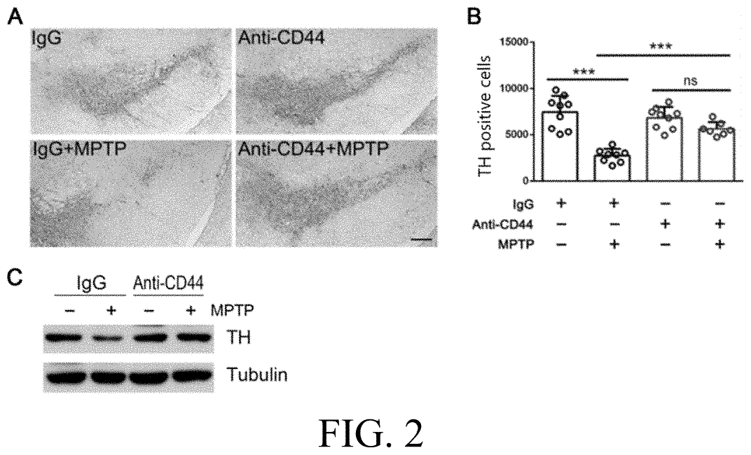

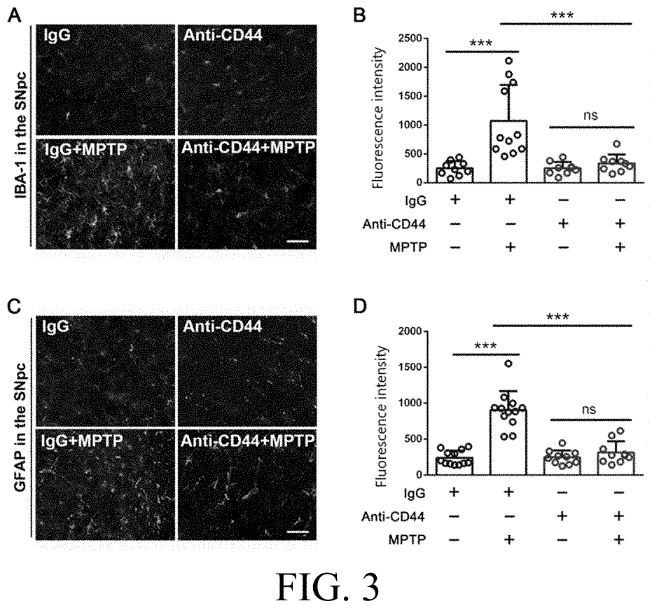

A CD44 antibody (Anti-CD44) is injected into the mouse cortex by a brain stereotactic technique. After the mouse recovers, a PD model is established by intraperitoneal injection with the neurotoxin MPTP (FIG. 1, panel A). The study finds that the CD44 antibody can significantly alleviate the motor dysfunction of PD model mice, for example, the rotarod time was significantly longer than that of the control MPTP group (FIG. 1, panel B), the stationary time in the tail suspension test is shorter than that of the control MPTP group (FIG. 1, panel C), the pole climbing time is shorter than that of the control MPTP group (FIG. 1, panel D), and the MPTP-induced olfactory dysfunction is improved (FIG. 1, panel D). The results of histomorphological analysis shows that the expression of tyrosine hydroxylase (TH) in the substantia nigra part of the PD model mice significantly decreases, while the expression of TH can be significantly increased by antibody treatment, indicating that the number of dopaminergic neurons is restored (FIG. 2, panels A and B). To further confirm the above results, the expression of TH protein is tested by a western blot method, and it is found that MPTP treatment results in a significant decrease in the amount of TH protein, and treatment with the antibody can reverse the downward trend (FIG. 2, panel C). In addition, the results of immunofluorescence analysis shows that the neuroinflammation in the substantia nigra part caused by MPTP is significantly improved by the antibody treatment, manifested as a decrease in the microglia activation marker, ionized calcium binding adapter molecule 1 (IBA-1) (FIG. 3, panels A and B), and the astrocyte activation marker, glial fibrillary acidic protein (GFAP) (FIG. 3, panels C and D).

1-2. (canceled)

3. A medicine for treating Parkinson's disease (PD), comprising a CD44 antibody.

4. A method for treating Parkinson's disease (PD), comprising administering a subject in need thereof a medicine comprising a CD44 antibody.

5. The method of claim 4, wherein the method relieves motor dysfunction, improves MPTP-induced olfactory dysfunction, and increases the expression of TH.
